(19) United States
(12) Patent Application Publication
(10) Pub. No.: US 2024/0197613 A1
(43) Pub. Date: Jun. 20, 2024

(54) Oral Care Compositions

(71) Applicant: Colgate-Palmolive Company, New York, NY (US)

(72) Inventors: Rong DONG, Highland Park, NJ (US); Gregory SZEWCZYK, Flemington, NJ (US); Suman CHOPRA, Monroe, NJ (US)

(73) Assignee: Colgate-Palmolive Company, New York, NY (US)

(21) Appl. No.: 18/543,423

(22) Filed: Dec. 18, 2023

Related U.S. Application Data
(60) Provisional application No. 63/476,037, filed on Dec. 19, 2022.

(51) Int. Cl. A61K 8/86; A61K 8/22; A61K 8/24; A61K 8/46; A61K 8/81; A61Q 11/00

(52) U.S. Cl. CPC A61K 8/86; A61K 8/22; A61K 8/24; A61K 8/463; A61K 8/8188; A61Q 11/00; A61K 2800/28

(57) ABSTRACT

Aspects of the invention are directed to oral care compositions having enhanced peroxide stability. In accordance with some aspects, provided is an oral care composition having a polyol; a whitening agent comprising a peroxide; a thickening agent; and an abrasive system comprising insoluble phosphate salts, wherein the oral care composition contains less than about 5 wt. % of water, and all weight percentages are based on the total weight of the oral care composition.

### CROSS-REFERENCE TO RELATED APPLICATIONS

This application claims the benefit of priority from U.S. Provisional Application No. 63/476,037, filed Dec. 19, 2022, the contents of which are hereby incorporated herein by reference in their entirety.

### BACKGROUND

Oral care compositions, such as dentifrice compositions, can typically include one or more ingredients for maintaining the health of the oral cavity. Consumers have become aware of and desiring oral care compositions that also maintain the aesthetics of their teeth. It can be desirable to have dentifrice compositions with a relatively high level of peroxide of at least about 1 wt. % of the total weight of the oral care composition to achieve high whitening efficacy. However, it can be challenging to formulate dentifrice compositions with a relatively high level of peroxide because peroxide can be reactive with other dentifrice components, such as fluoride, metal ions, abrasives, etc.

Oral care compositions containing a relatively high level of peroxide have been particularly difficult to formulate with abrasives. One approach to the formulation challenges associated with incorporating sufficient peroxide in a dentifrice composition can be to separately package and/or apply a peroxide separate from the dentifrice composition. Unfortunately, consumer compliance can be lower when a peroxide must be applied separately from the dentifrice composition.

As such, there is a need for an improved oral care composition having enhance peroxide stability.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to oral care compositions having enhanced peroxide stability. In accordance with some aspects, provided is an oral care composition having a polyol; a whitening agent comprising a peroxide; a thickening agent; and an abrasive based on insoluble phosphate salts, wherein the oral care composition contains less than about 5 wt. % of water, and all weight percentages are based on the total weight of the oral care composition.

According to further aspects, provided is an anhydrous oral care composition including a polyol comprising propylene glycol; a whitening agent comprising hydrogen peroxide; a thickening agent comprising an ammonium acryloyldimethyltaurate/VP copolymer, a sodium a acryloyldimethyltaurate, or a combination of two or more thereof; and an abrasive based on insoluble phosphate salts, wherein the oral care composition has about 7 wt. % or less of a total amount of polymers, based on the total weight of the oral care composition.

The peroxide of the oral care compositions disclosed herein may be selected from non-complexed peroxides including, e.g., hydrogen peroxide. Additionally or alternatively, the oral care compositions disclosed herein may, in some embodiments, have about 7 wt. % or less of a total amount of polymers.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the oral care composition. The symbol “°” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the oral care compositions of the instant disclosure can be free or essentially free of all components and elements positively recited throughout the instant disclosure. In some instances, the oral care compositions of the present disclosure may be substantially free of non-incidental amounts of the ingredient(s) or compound(s) described herein. A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the oral care composition includes both components (or the composition includes more than two components that overlap), an overlapping compound does not represent more than one component. For example, certain compounds may be characterized as both a fluoride ion source and a stannous ion source. If a particular claim to an oral care composition includes both a polyol and a humectant, glycerin will serve only as either a polyol or a humectant—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to oral care compositions having enhanced peroxide stability. Without being limited to any particular theory, it is believed that oral care compositions having a reduced and/or limited amount of polymers and certain combinations of thickening agents and abrasive(s) can simultaneously achieve enhanced availability of abrasives during application to an enamel surface and achieve significant improvements in peroxide stability.

In accordance with some aspects, provided is an oral care composition including a polyol; a whitening agent comprising a peroxide; a thickening agent; and an abrasive based on insoluble phosphate salts, wherein the oral care composition contains less than about 5 wt. % of water, and all weight percentages are based on the total weight of the oral care composition.

According to further aspects of the invention, provided is an anhydrous oral care composition including a polyol comprising propylene glycol; a whitening agent comprising hydrogen peroxide; a thickening agent comprising an ammonium acryloyldimethyltaurate/VP copolymer, a sodium acryloyldimethyltaurate, or a combination of two or more thereof; and an abrasive based on insoluble phosphate salts, wherein the oral care composition has about 7 wt. % or less of a total amount of polymers, based on the total weight of the oral care composition.

The oral care composition may be formulated to have about 7 wt. % or less of polymers selected from natural polymers, including gums and/or polysaccharides, and non-natural polymers, including acrylates, a polyitaconates, and/or acrylamides, and combinations of two or more thereof. For instance, in some embodiments, the oral care composition has about 7 wt. % or less of non-natural polymers, including acrylates, a polyitaconates, and/or acrylamides, based on the total weight of the oral care composition. In some embodiments, the total amount of non-natural polymers present in the oral care composition is about 6 wt. % or less, have about 5 wt. % or less, about 4 wt. % or less, about 3 wt. % or less, about 2 wt. % or less, about 1 wt. % or less, about 0.5 wt. % or less, based on the total weight of the oral care composition.

In other embodiments, the oral care composition has about 7 wt. % or less of natural polymers, including gums and polysaccharides, based on the total weight of the oral care composition. For example, the total amount of natural polymers present in the oral care composition may be about 6 wt. % or less, have about 5 wt. % or less, about 4 wt. % or less, about 3 wt. % or less, about 2 wt. % or less, about 1 wt. % or less, about 0.5 wt. % or less, based on the total weight of the oral care composition.

The oral care compositions may be formulated to have a total amount of all polymers of about 7 wt. % or less, based on the total weight of the oral care composition. For example, the total amount of all polymers present in the oral care composition may have about 6 wt. % or less, have about 5 wt. % or less, about 4 wt. % or less, about 3 wt. % or less, about 2 wt. % or less, about 1 wt. % or less, about 0.5 wt. % or less, based on the total weight of the oral care composition.

Additionally or alternatively, the oral care compositions typically have less than about 5 wt. % of water. In some embodiments, the amount of water present in the oral care composition is less than about 4 wt. %, less than about 3 wt. %, less than about 2 wt. %, less than about 1 wt. %, less than about 0.5 wt. %, less than about 0.1 wt. %, based on the total weight of the oral care composition. In some instances, the oral care composition is substantially free of or free of added water. In further instances, the oral care composition is substantially free of or free of water.

The oral care compositions may be in the form of a dentifrice, a toothpaste, a gel, and/or the like. Suitable components, such as those listed below, may be included or excluded from the formulations for the oral care compositions disclosed herein depending on the specific combination of other ingredients and/or the form of the oral care compositions. Additionally or alternatively, the oral care compositions may in some embodiments have a single phase, which contains that components and/or ingredients of the oral care composition. In other embodiments, the oral care composition may include two or more phases, such as two, three, four, or five phases.

The oral care compositions may include one or more polyol(s). The one or more polyol(s) may be present in the oral care composition in an amount from about 10 to about 90 wt. %, based on the total weight of the oral care composition. For example, the oral care composition may include polyol(s) in an amount from about 50 to about 90 wt. %, about 50 to about 85 wt. %, about 50 to about 80 wt. %, about 50 to about 75 wt. %, about 50 to about 70 wt. %, about 50 to about 67 wt. %, about 50 to about 64 wt. %, about 50 to about 61 wt. %, about 50 to about 58 wt. %; from about 55 to about 90 wt. %, about 55 to about 85 wt. %, about 55 to about 80 wt. %, about 55 to about 75 wt. %, about 55 to about 70 wt. %, about 55 to about 67 wt. %, about 55 to about 64 wt. %, about 55 to about 61 wt. %; from about 58 to about 90 wt. %, about 58 to about 85 wt. %, about 58 to about 80 wt. %, about 58 to about 75 wt. %, about 58 to about 70 wt. %, about 58 to about 67 wt. %, about 58 to about 64 wt. %; from about 60 to about 90 wt. %, about 60 to about 85 wt. %, about 60 to about 80 wt. %, about 60 to about 75 wt. %, about 60 to about 70 wt. %, about 60 to about 67 wt. %, about 60 to about 64 wt. %; from about 62 to about 90 wt. %, about 62 to about 85 wt. %, about 62 to about 80 wt. %, about 62 to about 75 wt. %, about 62 to about 70 wt. %; from about 64 to about 90 wt. %, about 64 to about 85 wt. %, about 64 to about 80 wt. %, about 64 to about 75 wt. %, about 64 to about 70 wt. %; from about 66 to about 90 wt. %, about 66 to about 85 wt. %, about 66 to about 80 wt. %, about 66 to about 75 wt. %, or any range or subrange thereof, based on the total weight of the oral care composition.

The polyol(s) may be chosen from glycols or compounds with numerous hydroxyl groups. The one or more polyols may be liquid at ambient temperature (25° C.). The polyol may be a humectant chosen from glycol, sorbitol, glycerin, inositol, maltitol, mannitol, xylitol, propylene glycol, polypropylene glycol (PPG), polyethylene glycol (PEG), a saccharide, polyoxypropylene-polyoxyethylene block copolymer and a combination of two or more thereof.

The polyol(s) may be selected from glycerin, glycol, inositol, maltitol, mannitol, sorbitol, xylitol, propylene glycol, polypropylene glycol (PPG), polyethylene glycol (PEG), a block copolymer of PPG and PEG, a saccharide (e.g., fructose, glucose, sucrose and mixtures of saccharides, such as honey), or a combination of two or more thereof. For instance, the oral care composition may comprise maltitol, mannitol, sorbitol, xylitol, a polypropylene glycol (PPG), a polyethylene glycol (PEG), a block copolymer of PPG and PEG, or a combination or two or more thereof.

In some cases, the oral care composition includes one or more polyol selected from the group consisting of C₂-C₃₂ polyols. The one or more polyols may have from 2 to 32 carbon atoms, from 3 to 16 carbon atoms, or from 3 to 12 carbon atoms. For example, the oral care composition may comprise ethylene glycol, propylene glycol, butylene glycol, hexylene glycol, glycerin, diglycerin, diethylene glycol, and dipropylene glycol, or a combination of two or more thereof. Additional, non-limiting examples of polyols that may, optionally, be incorporated in the oral care composition include alkanediols, such as glycerin, 1,2,6-hexanetriol, trimethylolpropane, ethylene glycol, propylene glycol, butylene glycol, pentylene glycol, hexylene glycol, diethylene glycol, triethylene glycol, tetracthylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers, such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture thereof.

Additionally or alternatively, the oral care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof. In some embodiments, the polyol is a polyethylene glycol, such as polyethylene glycol 600 (CAS-25322-68-3). In some embodiments, the oral care compositions comprise one or more polyethylene glycols, e.g., polyethylene glycols in a molecular weight range from 200 to 800. For example, the oral care composition may comprise polyethylene glycol 200, polyethylene glycol 300, polyethylene glycol 400, polyethylene glycol 600, polyethylene glycol 800, or a combination of two or more thereof.

Typically, the oral care compositions include one or more whitening agent comprising a peroxide, preferably in an effective amount. The amount of whitening agent(s) present in the oral care composition may be from about 1 to about 30 wt. %, based on the total weight of the oral care composition. For example, the whitening agent(s) may be present in the oral care composition in an amount from about 1 to about 30 wt. %, about 1 to about 26 wt. %, about 1 to about 22 wt. %, about 1 to about 19 wt. %, about 1 to about 16 wt. %, about 1 to about 13 wt. %, about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 30 wt. %, about 2 to about 26 wt. %, about 2 to about 22 wt. %, about 2 to about 19 wt. %, about 2 to about 16 wt. %, about 2 to about 13 wt. %, about 2 to about 10 wt. %, about 2 to about 8 wt. %, about 2 to about 6 wt. %, about 2 to about 5 wt. %, about 2 to about 4 wt. %; from about 3 to about 30 wt. %, about 3 to about 26 wt. %, about 3 to about 22 wt. %, about 3 to about 19 wt. %, about 3 to about 16 wt. %, about 3 to about 13 wt. %, about 3 to about 10 wt. %, about 3 to about 8 wt. %, about 3 to about 6 wt. %; from about 5 to about 30 wt. %, about 5 to about 26 wt. %, about 5 to about 22 wt. %, about 5 to about 19 wt. %, about 5 to about 16 wt. %, about 5 to about 13 wt. %, about 5 to about 10 wt. %, about 5 to about 8 wt. %; from about 8 to about 30 wt. %, about 8 to about 26 wt. %, about 8 to about 22 wt. %, about 8 to about 19 wt. %, about 8 to about 16 wt. %, about 8 to about 13 wt. %; from about 11 to about 30 wt. %, about 11 to about 26 wt. %, about 11 to about 22 wt. %, about 11 to about 19 wt. %, about 11 to about 16 wt. %; from about 14 to about 30 wt. %, about 14 to about 26 wt. %, about 14 to about 22 wt. %, about 14 to about 19 wt. %; from about 18 to about 30 wt. %, about 18 to about 26 wt. %, about 18 to about 22 wt. %, or any range or subrange thereof, based on the total weight of the oral care composition.

The whitening agent(s) typically comprise a peroxide, such as hydrogen peroxide, peroxides of alkali and alkaline earth metals, organic peroxy compounds, peroxy acids, salts thereof, or combinations of two or more thereof. Peroxides of alkali and alkaline earth metals include lithium peroxide, potassium peroxide, sodium peroxide, magnesium peroxide, calcium peroxide, barium peroxide, and a combination of two or more thereof. Examples of organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, monoperoxyphthalate, or a combination of two or more thereof. Peroxy acids and their salts include organic peroxy acids, such as alkyl peroxy acids, monoperoxyphthalate, and a combination thereof as well as inorganic peroxy acid salts, such as persulfate, dipersulfate, percarbonate, perphosphate, perborate and persilicate salts of alkali and/or alkaline earth metals, such as lithium, potassium, sodium, magnesium, calcium and barium, and mixtures thereof. In various embodiments, the peroxide compound comprises hydrogen peroxide, urea peroxide, sodium percarbonate, or a combination of two or more thereof.

The one or more peroxide(s) may be a non-complexed peroxide. The non-complexed peroxide may be chosen from one or more of the peroxides described above. In some preferred embodiments, the non-complexed peroxide comprises hydrogen peroxide.

The total amount of whitening agent may comprise from about 0.01 to about 10 wt. % of a non-complexed peroxide source, based on the total weight of the oral care composition. For example, the whitening agent may comprise a non-complexed peroxide source in an amount from about 0.01 to about 10 wt. %, about 0.01 to about 8 wt. %, about 0.01 to about 6 wt. %, about 0.01 to about 4 wt. %, about 0.01 to about 3 wt. %, about 0.01 to about 2 wt. %, about 0.01 to about 1 wt. %; from about 0.1 to about 10 wt. %, about 0.1 to about 8 wt. %, about 0.1 to about 6 wt. %, about 0.1 to about 4 wt. %, about 0.1 to about 3 wt. %, about 0.1 to about 2 wt. %, about 0.1 to about 1 wt. %; from about 0.5 to about 10 wt. %, about 0.5 to about 8 wt. %, about 0.5 to about 6 wt. %, about 0.5 to about 4 wt. %, about 0.5 to about 3 wt. %, about 0.5 to about 2 wt. %, about 0.5 to about 1 wt. %; from about 1 to about 10 wt. %, about 1 to about 8 wt. %, about 1 to about 6 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 4 wt. %; from about 3 to about 10 wt. %, about 3 to about 8 wt. %, about 3 to about 6 wt. %, about 3 to about 4 wt. %; from about 4 to about 10 wt. %, about 4 to about 8 wt. %, about 4 to about 6 wt. %, or any range or subrange thereof, based on the total weight of the oral care composition.

In some embodiments, however, the whitening agent may comprise a peroxide complex.

The peroxide complex may comprise a peroxide component and a porous cross-linked polymer. As referred to herein, a “peroxide component” is any oxidizing compound comprising a bivalent oxygen-oxygen group. Peroxide components include peroxides and hydroperoxides disclosed above, such as hydrogen peroxide, peroxides of alkali and alkaline earth metals, organic peroxy compounds, peroxy acids, salts thereof, and mixtures thereof. In various embodiments, the peroxide component comprises hydrogen peroxide, urea peroxide, sodium percarbonate and mixtures thereof. In one embodiment, the peroxide component comprises hydrogen peroxide. In one embodiment, the peroxide component consists essentially of hydrogen peroxide.

The whitening agent may comprise a peroxide complex, such as PVP complexed with hydrogen peroxide, cross-linked PVP complexed with hydrogen peroxide, silica complexed with hydrogen peroxide, tetrasodium pyrophosphate complexed with hydrogen peroxide, or a combination of two or more thereof. The peroxide complex may, also or alternatively, be selected from N-vinyl heterocyclic polymers. Preferably, the N-vinyl heterocyclic polymer is crosslinked and adsorbs, absorbs, complexes, or otherwise retains the peroxide component. The chemical and physical characteristics of the particulate hinder the release of the peroxide compound from the polymer particulates, and in some embodiments provides controlled release of the peroxide compound. The peroxide complex preferably comprises a peroxide component at a level of from about 0.1 to about 25 wt. %, from about 1 to about 25 wt. %, from about 5 to about 18 wt. %, based on the weight of the peroxide complex. In some preferred embodiments, the oral care composition comprises a complex of peroxide adsorbed to cross-linked polyvinylpyrrollidone.

In some embodiments, a non-peroxide whitening agent may be included in the oral care composition. Whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium and barium.

Non-peroxide whitening agents also include colorants, such as titanium dioxide and hydroxyapatite, pigments, or dyes.

The oral care composition typically includes a thickening agent in an amount, e.g., from about 0.1 to about 20 wt. %, based on the total weight of the oral care composition. For instance, the total amount from about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.8 wt. %, about 0.1 to about 0.5 wt. %; from about 0.3 to about 7 wt. %, about 0.3 to about 5 wt. %, about 0.3 to about 4 wt. %, about 0.3 to about 3 wt. %, about 0.3 to about 2.5 wt. %, about 0.3 to about 2 wt. %, about 0.3 to about 1 wt. %, about 0.3 to about 0.8 wt. %; from about 0.5 to about 7 wt. %, about 0.5 to about 5 wt. %, about 0.5 to about 4 wt. %, about 0.5 to about 3 wt. %, about 0.5 to about 2.5 wt. %, about 0.5 to about 2 wt. %, about 0.5 to about 1 wt. %; from about 0.8 to about 7 wt. %, about 0.8 to about 5 wt. %, about 0.8 to about 4 wt. %, about 0.8 to about 3 wt. %, about 0.3 to about 2.5 wt. %, about 0.8 to about 2 wt. %; from about 1 to about 7 wt. %, about 1 to about 5 wt. %, about 1 to about 4 wt. %, about 1 to about 3 wt. %, about 1 to about 2.5 wt. %, about 1 to about 2 wt. %; from about 1.5 to about 7 wt. %, about 1.5 to about 5 wt. %, about 1.5 to about 4 wt. %, about 1.5 to about 3 wt. %, about 1.5 to about 2.5 wt. %; from about 2.5 to about 7 wt. %, about 2.5 to about 5 wt. %, about 2.5 to about 4 wt. %; from about 4 to about 7 wt. %, about 4 to about 5 wt. %; from about 5 to about 20 wt. %, about 5 to about 15 wt. %, about 5 to about 10 wt. %, about 5 to about 7 wt. %; from about 8 to about 20 wt. %, about 8 to about 15 wt. %, about 8 to about 10 wt. %; from about 11 to about 20 wt. %, about 11 to about 15 wt. %, about 8 to about 10 wt. %; from about 14 to about 20 wt. %, about 14 to about 17 wt. %, or any range or subrange thereof, based on the total weight of the oral care composition.

The oral care composition may comprise a thickening system formed from one or more thickening agents. In some embodiments, the one or more thickening agent(s) may be selected from an ammonium acryloyldimethyltaurate/VP copolymer, a sodium polyacryloyldimethyltaurate, a polyvinylpyrrolidone, a cross-polymer 6, and a combination of two or more thereof. In at least one preferred embodiment, the thickening agent(s) comprises an ammonium acryloyldimethyltaurate/VP copolymer, a sodium acryloyldimethyltaurate, or a combination of two or more thereof.

Sodium acryloyldimethyltaurate, as used herein, includes sodium polyacryloyldimethyltaurate. The sodium acryloyldimethyltaurate may be chosen from those having a molecular weight of from about 100,000 to about 10,000,000 g/mol. For example, the thickening system may comprise a sodium acryloyldimethyltaurate having a molecular weight of about 100,000 to about 10,000,000 g/mol, about 100,000 to about 8,000,000 g/mol, about 100,000 to about 6,000,000 g/mol, about 100,000 to about 4,000,000 g/mol, about 100,000 to about 2,000,000 g/mol, about 100,000 to about 1,000,000 g/mol; from about 250,000 to about 10,000,000 g/mol, about 250,000 to about 8,000,000 g/mol, about 250,000 to about 6,000,000 g/mol, about 250,000 to about 4,000,000 g/mol, about 250,000 to about 2,000,000 g/mol, about 250,000 to about 1,000,000 g/mol; from about 500,000 to about 10,000,000 g/mol, about 500,000 to about 8,000,000 g/mol, about 500,000 to about 6,000,000 g/mol, about 500,000 to about 4,000,000 g/mol, about 500,000 to about 2,000,000 g/mol, about 500,000 to about 1,000,000 g/mol; from about 1,000,000 to about 10,000,000 g/mol, about 1,000,000 to about 8,000,000 g/mol, about 1,000,000 to about 6,000,000 g/mol, about 1,000,000 to about 4,000,000 g/mol, about 1,000,000 to about 2,000,000 g/mol; from about 2,000,000 to about 10,000,000 g/mol, about 2,000,000 to about 8,000,000 g/mol, about 2,000,000 to about 6,000,000 g/mol, about 2,000,000 to about 4,000,000 g/mol; from about 4,000,000 to about 10,000,000 g/mol, about 4,000,000 to about 8,000,000 g/mol, about 4,000,000 to about 6,000,000 g/mol; from about 6,000,000 to about 10,000,000 g/mol, about 6,000,000 to about 8,000,000 g/mol, or any range or subrange thereof.

The ammonium acryloyldimethyltaurate/VP copolymer and/or the cross linked polyvinylpyrrolidone may be chosen from those having a molecular weight of from about 100,000 to about 10,000,000 g/mol. For example, the ammonium acryloyldimethyltaurate/VP copolymer and/or the cross linked polyvinylpyrrolidone may have a molecular weight of about 100,000 to about 10,000,000 g/mol, about 100,000 to about 8,000,000 g/mol, about 100,000 to about 6,000,000 g/mol, about 100,000 to about 4,000,000 g/mol, about 100,000 to about 2,000,000 g/mol, about 100,000 to about 1,000,000 g/mol; from about 250,000 to about 10,000,000 g/mol, about 250,000 to about 8,000,000 g/mol, about 250,000 to about 6,000,000 g/mol, about 250,000 to about 4,000,000 g/mol, about 250,000 to about 2,000,000 g/mol, about 250,000 to about 1,000,000 g/mol; from about 500,000 to about 10,000,000 g/mol, about 500,000 to about 8,000,000 g/mol, about 500,000 to about 6,000,000 g/mol, about 500,000 to about 4,000,000 g/mol, about 500,000 to about 2,000,000 g/mol, about 500,000 to about 1,000,000 g/mol; from about 1,000,000 to about 10,000,000 g/mol, about 1,000,000 to about 8,000,000 g/mol, about 1,000,000 to about 6,000,000 g/mol, about 1,000,000 to about 4,000,000 g/mol, about 1,000,000 to about 2,000,000 g/mol; from about 2,000,000 to about 10,000,000 g/mol, about 2,000,000 to about 8,000,000 g/mol, about 2,000,000 to about 6,000,000 g/mol, about 2,000,000 to about 4,000,000 g/mol; from about 4,000,000 to about 10,000,000 g/mol, about 4,000,000 to about 8,000,000 g/mol, about 4,000,000 to about 6,000,000 g/mol; from about 6,000,000 to about 10,000,000 g/mol, about 6,000,000 to about 8,000,000 g/mol, or any range or subrange thereof.

The oral care composition may include one or more additional thickening agent(s) including those selected from, e.g., polysaccharides, acrylates, polymers, polyitaconates, acrylamides, and combinations of two or more thereof. Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, carrageenan, xanthan gum, or a combination of two more thereof.

Examples of acrylates include, e.g., isobutyl acrylate, tert-butyl acrylate, 2-ethylhexyl acrylate, lauryl acrylate, lauryl/tridecyl acrylate, cetyl acrylate, stearyl acrylate, cyclohexyl acrylate, benzyl acrylate, isobornyl acrylate, 2-methoxyethyl acrylate, 2-ethoxyethyl acrylate, 2-ethoxyethoxyethyl acrylate, 2-phenoxyethyl acrylate, tetrahydrofurfuryl acrylate, 2-hydroxyethyl acrylate, 2-hydroxypropyl acrylate, 4-hydroxybutyl acrylate, dimethylaminoethyl acrylate, 1,4-butanediol acrylate, or a combination of two or more thereof.

The acrylate may be chosen from diacrylates. In some embodiments, the oral care composition includes a diacrylate chosen from 1,4-butanediol, 1,6-hexanediol, tetraethylene glycol, tripropylene glycol, ethoxylated bisphenol-A, and a combination of two or more thereof. Triacrylate monomers include trimethylol propane, ethoxylated, glyceryl propoxy, and pentaerythritol.

Acrylates further include methacrylates, such as methyl methacrylate, ethyl methacrylate, n-butyl methacrylate, isobutyl methacrylate, tert-butyl methacrylate, 2-ethylhexyl methacrylate, lauryl methacrylate, alkyl methacrylate, tridecyl methacrylate, stearyl methacrylate, cyclohexyl methacrylate, benzyl methacrylate, isobornyl methacrylate, 2-hydroxyethyl methacrylate, 2-hydroxypropyl methacrylate, dimethylaminocthyl methacrylate, diethylaminoethyl methacrylate, glycidyl methacrylate, tetrahydrofurfuryl methacrylate, allyl methacrylate, ethylene glycol methacrylate, tricthylene glycol methacrylate, tetracthylene glycol methacrylate, 1,3-butyleneglycol methacrylate, 1,6-hexanediol methacrylate, trimethylopropane methacrylate, ethoxyethyl methacrylate, trifluoroethyl methacrylate, or a combination of two or more thereof.

Examples of acrylamides include, but are not limited to, acrylamide, methacrylamide and di(C₁-C₃₀) alkyl-acrylamides and -methacrylamides such as those of methyl, ethyl, propyl, butyl, pentyl, hexyl and the like. N-substituted acrylamides that may be suitable include N-ethylacrylamide, N-tert-butylacrylamide, N-tert-octylacrylamide, N-octylacrylamide, N-decylacrylamide, N-dodecylacrylamide and the corresponding N-substituted methacrylamides. Other N-substituted acrylamides include N-hydroxymethyl acrylamide, N-isopropylacrylamide, N-methylacrylamide, N,N′-methylenebisacrylamide, N-isobutoxymethylacrylamide, N,N-dimethylacrylamide, 2-acrylamido-2-methylpropanesulfonic acid, or a combination of two or more thereof.

The oral care composition may include one or more abrasive, e.g., in an amount from about 1 to about 35 wt. %, based on the total weight of the oral care composition. In some instances, the total amount of abrasive(s) present in the oral care composition is from about 1 to about 35 wt. %, about 1 to about 35 wt. %, about 1 to about 31 wt. %, about 1 to about 27 wt. %, about 1 to about 23 wt. %, about 1 to about 19 wt. %, about 1 to about 17 wt. %, about 1 to about 15 wt. %, about 1 to about 13 wt. %; from about 4 to about 35 wt. %, about 4 to about 35 wt. %, about 4 to about 31 wt. %, about 4 to about 27 wt. %, about 4 to about 23 wt. %, about 4 to about 19 wt. %, about 4 to about 17 wt. %, about 4 to about 15 wt. %, about 4 to about 13 wt. %; from about 7 to about 35 wt. %, about 7 to about 35 wt. %, about 7 to about 31 wt. %, about 7 to about 27 wt. %, about 7 to about 23 wt. %, about 7 to about 19 wt. %, about 7 to about 17 wt. %, about 7 to about 15 wt. %, about 7 to about 13 wt. %; from about 10 to about 35 wt. %, about 10 to about 35 wt. %, about 10 to about 31 wt. %, about 10 to about 27 wt. %, about 10 to about 23 wt. %, about 10 to about 19 wt. %, about 10 to about 17 wt. %, about 10 to about 15 wt. %; from about 13 to about 35 wt. %, about 13 to about 35 wt. %, about 13 to about 31 wt. %, about 13 to about 27 wt. %, about 13 to about 23 wt. %, about 13 to about 19 wt. %, about 13 to about 17 wt. %, about 13 to about 15 wt. %; from about 15 to about 35 wt. %, about 15 to about 35 wt. %, about 15 to about 31 wt. %, about 15 to about 27 wt. %, about 15 to about 23 wt. %, about 15 to about 19 wt. %; from about 17 to about 35 wt. %, about 17 to about 35 wt. %, about 17 to about 31 wt. %, about 17 to about 27 wt. %, about 17 to about 23 wt. %; from about 19 to about 35 wt. %, about 19 to about 35 wt. %, about 19 to about 31 wt. %, about 19 to about 27 wt. %, about 19 to about 23 wt. %, or any range or subrange thereof, based on the total weight of the oral care composition.

The abrasive(s) based on insoluble phosphate salts may be chosen from calcium pyrophosphate, dicalcium orthophosphate dihydrate, tricalcium phosphate, and calcium polymetaphosphate, and a combination of two or more thereof. The total amount of abrasives is preferably comprised primarily of calcium pyrophosphate and, in at least one embodiment, calcium pyrophosphate is the sole abrasive in the oral care composition. For example, the calcium pyrophosphate may comprise about 50 wt. % or more, about 60 wt. % or more, about 70 wt. % or more, about 80 wt. % or more, about 95 wt. % or more, about 98 wt. % or more, or about 99 wt. % or more of the total amount of the abrasive agent in the oral care composition.

The oral care compositions may include one or more surfactant(s) in various amounts. The oral care composition may comprise one or more anionic surfactant, one or more cationic surfactant, one or more nonionic surfactant, or a combination of two or more thereof. For example, the oral care composition may include one or more anionic surfactant(s) in an amount of about 0.5 to about 10 wt. %, based on the total weight of the oral care composition. In some instances, the anionic surfactant(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The anionic surfactant(s) may be selected from sulfate based anionic surfactants and non-sulfate based anionic surfactants. For example, the oral care composition may include an anionic surfactant chosen from ammonium lauryl sulfate, ammonium lauryl ether sulfate, sodium dodecyl sulfate, sodium coco-sulfate, ammonium coco-sulfate, and a combination of two or more thereof. In some preferred embodiments, the oral care composition comprises sodium lauryl sulfate, sodium lauryl ether sulfate, or a combination thereof. In further embodiments, one or more anionic surfactant is selected from sodium lauryl sulfate, sodium lauryl ether sulfate, sodium lauryl sarcosinate, sodium cocoyl methyl taurate, sodium monoglyceride sulfate, sodium cetaryl sulfate, potassium cocoyl glycinate, sodium lauryl phosphate, sodium lauryl lactylate, sodium lauryl sulfoacetate, sodium lauryl glutamate, sodium lauryl isethionate, sodium laureth carboxylate, sodium dodecyl benzenesulfonate, and a combination of two or more thereof.

The one or more anionic surfactant(s) may be selected from non-sulfate based anionic surfactants, such as sulfonated monoglycerides of fatty acids, acyl isethionates, acyl sarcosinates, acyl taurate, and a combination of two or more thereof. Non-limiting examples of acyl isethionates include sodium isethionate, sodium cocoyl isethionate, sodium lauroyl methyl isethionate, and sodium cocoyl methyl isethionate. Sulfonated monoglycerides of fatty acids include sodium coconut monoglyceride sulfonates and the like. Examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, ammonium lauroyl sarcosinate, and combinations of two or more thereof. Examples of acyl taurate surfactants include sodium cocoyl taurate, potassium cocoyl taurate, potassium methyl cocoyl taurate, sodium caproyl methyl taurate, sodium cocoyl taurate, sodium lauroyl taurate, sodium methyl cocoyl taurate (SMCT), sodium methyl lauroyl taurate, sodium methyl myristoyl taurate, sodium methyl oleoyl taurate, sodium methyl palmitoyl taurate, sodium methyl stearoyl taurate, and combinations of two or more thereof. In some embodiments, the oral care composition comprises sodium lauroyl methyl taurate (or sodium methyl lauroyl taurate), sodium methyl cocoyl taurate (SMCT), or a combination thereof.

The anionic surfactant(s) may have at least one acyl group, preferably, comprising a carbon chain of 8 to 21 carbons. In some instance, the alkyl group(s) of the anionic surfactant(s) comprise a carbon chain of 8 to 19 carbons, 8 to 17 carbons, 8 to 15 carbons, 8 to 13 carbons, 8 to 11 carbons; 9 to 21 carbons, 9 to 19 carbons, 9 to 17 carbons, 9 to 15 carbons, 9 to 13 carbons, 9 to 11 carbons; 11 to 21 carbons, 11 to 19 carbons, 11 to 17 carbons, 11 to 15 carbons, 11 to 13 carbons; 13 to 21 carbons, 13 to 19 carbons, 13 to 17 carbons, or any range or subrange thereof. The anionic surfactants disclosed herein may be incorporated in a salt form. The salt form of the anionic surfactants may have an alkali metal (e.g., sodium or potassium) and/or ammonium group.

Additionally or alternatively, the oral care composition may comprise one or more nonionic surfactant(s). The oral care composition may include nonionic surfactant(s) in an amount of about 0.5 to about 10 wt. %, based on the total weight of the oral care composition. In some instances, the nonionic surfactant(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The one or more nonionic surfactant(s) may be selected from glucosides, compounds produced by the condensation of alkylene oxide groups (hydrophilic in nature) with an organic hydrophobic compound, which may be aliphatic or alkylaromatic in nature. Examples of glucoside surfactants include decyl glucoside, stearyl glucoside, lauryl glucoside, coco-glucoside, cetearyl glucoside, decyl lauryl glucoside, lauroyl ethyl glucoside, myristoyl ethyl glucoside, oleoyl ethyl glucoside, or a combination of two or more thereof. In some embodiments, the surfactant system includes one or more nonionic surfactant(s) chosen from lauryl glucoside, lauroyl ethyl glucoside, myristoyl ethyl glucoside, oleoyl ethyl glucoside, and a combination of two or more thereof. Additionally or alternatively, the glucoside surfactant may be chosen from polyglucosides, such as alkylpolyglucosides.

Further examples of nonionic surfactants include poloxamers, polyethylene oxide condensates of alkyl phenols, products derived from the condensation of ethylene oxide with the reaction product of propylene oxide and ethylene diamine, ethylene oxide condensates of aliphatic alcohols, acids, and esters, long chain tertiary amine oxides, long chain tertiary phosphine oxides, long chain dialkyl sulfoxides and mixtures of such materials. Additional examples of nonionic surfactants include polyoxyethylene, polyoxyethylene sorbitan esters, polyoxyl n₄₀₋₆₀ hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, or a combination of two or more thereof. In some instances, the nonionic surfactant comprises amine oxides, fatty acid amides, ethoxylated fatty alcohols, block copolymers of polyethylene glycol and polypropylene glycol, glycerol alkyl esters, polyoxyethytene glycol octylphenol ethers, sorbitan alkyl esters, polyoxyethylene glycol sorbitan alkyl esters, or a combination of two or more thereof.

In some embodiments, the oral care composition may comprise one or more cationic surfactant(s). The oral care composition may include cationic surfactant(s) in an amount of about 0.5 to about 10 wt. %, based on the total weight of the oral care composition. In some instances, the cationic surfactant(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

Examples of cationic surfactants that may be present in the oral care composition include cetyl pyridinium chloride, coamidopropyl PG dimonium chloride phosphate (Phospholipid CDM), myristylamidopropyl PG dimonium chloride phosphate (Phospholipid PTM), stearamidopropyl PG dimonium chloride phosphate (Phospholipid SV), steapyrium chloride (Catemol WPC), and other suitable cationic materials. The cationic surfactants may be present in the form of a salt.

The oral care compositions may include an anti-calculus agent. The amount of anti-calculus agent(s) present in the oral care composition may be from about 0.5 to about 10 wt. %, based on the total weight of the oral care composition. In some instances, the anti-calculus agent(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The oral care compositions may include anti-calculus agents chosen from phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates, and combinations of two or more thereof. Examples of additional anti-calculus agents include tetrasodium pyrophosphate, ammonium tripolyphosphate, hexametaphosphate salt, polycarboxylate polymers, polyvinyl methyl ether/maleic anhydride (PVME/MA) copolymers, or combinations of two or more thereof. In some embodiments, the oral care composition includes one or more alkali metal polyphosphate, such as tetrasodium pyrophosphate.

The oral care compositions may include a fluoride ion source. The fluoride ion source may be present in an effective amount. In some cases, the fluoride ion source is present in the oral care composition in an amount of about 0.1 to about 7 wt. %, based on the total weight of the oral care composition. For example, the fluoride ion source may be present in an amount of about 0.1 to about 7 wt. %, about 0.1 to about 5 wt. %, about 0.1 to about 3 wt. %, about 0.1 to about 2 wt. %, about 0.1 to about 1 wt. %; from about 0.4 to about 7 wt. %, about 0.4 to about 5 wt. %, about 0.4 to about 3 wt. %, about 0.4 to about 2 wt. %, about 0.4 to about 1 wt. %; from about 0.8 to about 7 wt. %, about 0.8 to about 5 wt. %, about 0.8 to about 3 wt. %, about 0.8 to about 2 wt. %, about 0.8 to about 1.5 wt. %; from about 1.4 to about 7 wt. %, about 1.4 to about 5 wt. %, about 1.4 to about 3 wt. %; from about 2 to about 7 wt. %, about 2 to about 5 wt. %, about 2 to about 3 wt. %; from about 3 to about 7 wt. %, about 3 to about 5 wt. %, about 3 to about 4 wt. %; from about 4 to about 7 wt. %, about 4 to about 6 wt. %, about 4 to about 5 wt. %; from about 5 to about 7 wt. %, about 5 to about 6 wt. %, or any range or subrange thereof, based on the total weight of the oral care composition.

The fluoride ion source may be selected from soluble fluoride ion salts. For example, the fluoride ion source may comprise sodium fluoride, potassium fluoride, calcium fluoride, zinc fluoride, zinc ammonium fluoride, lithium fluoride, ammonium fluoride, stannous fluoride, stannous fluorozirconate, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, amine fluorides or a combination of two or more thereof. In some embodiments, the fluoride ion source comprises sodium fluoride, sodium monofluorophosphate, or a combination thereof. Additional examples of fluoride ion sources are disclosed in U.S. Pat. Nos. 3,535,421, 4,885,155, and 3,678,154, the disclosures of which are hereby incorporated by reference in their entirety for all purposes.

The oral care compositions may include a desensitizing agent. The amount of desensitizing agent may be from about 0.5 to about 9 wt. %, based on the total weight of the oral care composition. For example, the amount of desensitizing agent present in the oral care composition may be from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 9 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 9 wt. %, about 2 to about 8 wt. %, about 2 to about 6 wt. %, about 2 to about 5 wt. %, about 2 to about 4 wt. %, about 2 to about 3 wt. %; from about 3 to about 9 wt. %, about 3 to about 8 wt. %, about 3 to about 6 wt. %, about 3 to about 5 wt. %, about 3 to about 4 wt. %; from about 4 to about 9 wt. %, about 4 to about 8 wt. %, about 4 to about 6 wt. %, about 4 to about 5 wt. %; from about 5 to about 9 wt. %, about 5 to about 8 wt. %, about 5 to about 6 wt. %; from about 6 to about 9 wt. %, about 6 to about 8 wt. %; from about 7 to about 9 wt. %, about 7 to about 8 wt. %, about 8 to about 10 wt. %, or any range or subrange therefrom, based on the total weight of the oral care composition.

The desensitizing agent(s) may comprise, e.g., benzocaine, potassium nitrate; potassium fluoride; strontium chloride; potassium chloride; potassium citrate; iron oxalate; sodium nitrate; lithium nitrate; magnesium nitrate; calcium nitrate; calcium hydroxide; dibasic calcium phosphate; strontium acetate; sodium monofluorophosphate; bisabolol; a local or systemic analgesic agent such as NSAIDS, aspirin, acetaminophen and/or codeine; or combinations of two or more thereof. For example, the desensitizing agent(s) may be chosen from potassium nitrate, potassium citrate, potassium chloride, potassium bicarbonate, potassium oxalate, and a combination of two or more thereof. In some embodiments, the desensitizing agent is potassium nitrate.

In some embodiments, the oral care compositions may include one or more sweeteners.

The oral care composition may include caloric sweeteners and/or non-caloric sweeteners. Examples of non-caloric sweeteners include saccharin, e.g., sodium saccharin, acesulfame, neotame, cyclamate or sucralose; natural high-intensity sweeteners, such as thaumatin, stevioside or glycyrrhizin; or sugar alcohols, such as sorbitol, xylitol, maltitol and mannitol. Examples of caloric sweeteners include sugars, such as fructose, glucose, sucrose, and high fructose syrups.

The one or more sweetener(s) may be present in the oral care composition in an amount from about 0.1 to about 7 wt. %, based on the total weight of the oral care composition. For example, the oral care composition may have a total amount of sweetener(s) from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 7 wt. %, about 1 to about 5 wt. %, about 1 to about 4 wt. %, about 1 to about 3 wt. %, about 1 to about 2 wt. %; from about 2 to about 7 wt. %, about 2 to about 5 wt. %, about 2 to about 4 wt. %, about 2 to about 3 wt. %; from about 3 to about 7 wt. %, about 3 to about 5 wt. %, about 3 to about 4 wt. %; from about 4 to about 7 wt. %, about 4 to about 5 wt. %; from about 5 to about 7 wt. %, or any range or subrange thereof, based on the total weight of the oral care composition.

In some embodiments, the oral care composition preferably is substantially free or free of caloric sweeteners. For example, the oral care compositions may have about 4 wt. % or less, about 3 wt. % or less, about 2 wt. % or less, about 1 wt. % or less, about 0.5 wt. % or less, or about 0.1 wt. % or less, based on the weight of the oral care composition. In at least one embodiment, the oral care composition contains about 0 wt. % or 0 wt. % of caloric sweeteners, based on the weight of the oral care composition.

The oral care compositions of the present disclosure may include a flavoring agent. The flavoring agent is typically incorporated in the oral care composition at a concentration of about 0.01 to about 3 wt. % by weight of the oral care composition. For example, the amount of flavoring agent(s) present in the oral care composition may be from about 0.01 to about 2 wt. %, about 0.01 to about 1 wt. %, about 0.01 to about 0.5 wt. %, about 0.01 to about 0.1 wt. %; from about 0.1 to about 3 wt. %, about 0.1 to about 2 wt. %, about 0.1 to about 1 wt. %, about 0.1 to about 0.5 wt. %; from about 0.5 to about 3 wt. %, about 0.5 to about 2 wt. %, about 0.5 to about 1 wt. %; from about 1 to about 3 wt. %, about 1 to about 2 wt. %; from about 2 to about 3 wt. %, including any range or subrange thereof, based on the total weight of the oral care composition.

Suitable flavoring agents include, but are not limited to, essential oils and various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Additional flavoring agents may include, but are not limited to menthol, artificial vanilla, cinnamon derivatives, various fruit flavors, spearmint oil, peppermint oil, cinnamon oil, oil of wintergreen (methylsalicylate), clove oil, bay oil, anise oil, eucalyptus oil, thyme oil, cedar leaf oil, oil of nutmeg, oil of sage, oil of bitter almonds, cassia oil, and a combination of two or more thereof.

The oral care compositions may include one or more colorants. Exemplary colorants can include natural or uncertified colors from natural sources or certified colors for the effect of color. In some embodiments, the colorant can include dyes, certified aluminum lakes or colors derived from a natural source. The colorant may be water-based, oil-based or dry. The colorants can be primary colors, blends of colors or discrete mixtures of colors, such as confetti. The concentrations of the colorant in the oral care composition may be from trace amount to about 0.6 wt. %, from about 0.1 to about 0.5 wt. %, about 0.2 to about 0.4 wt. %, or about 0.15 to about 0.35 wt. %, based on the total weight of the oral care composition.

The oral care composition may include one or more pH adjusters to increase or decrease the overall pH of the oral care composition. For example, one or more acids may be included to decrease the pH of the oral care composition. Examples of suitable acids for decreasing the pH of the oral care composition include, but are not limited to, citric acid, acetic acid, and the like. The oral care composition may include one or more bases, such as sodium hydroxide, potassium hydroxide and the like, to increase the pH of the oral care composition. Additional or alternative acids and bases that are suitable for adjusting the pH of the oral care composition are readily known to one of ordinary skill in the art.

The amount of the pH adjuster in the oral care composition may be based on the desired pH of the final oral care composition and/or product. For example, the total amount of the pH adjuster may range from about 0.05 to about 20 wt. %, based on the total weight of the oral care composition. In some instances, the total amount of pH adjuster is from about 0.05 to about 15 wt. %, about 0.1 to about 10 wt. %, or about 0.12 to about 5 wt. %, including ranges and sub-ranges thereof, based on the total weight of the oral care composition.

In another aspect, the present disclosure provides a method of using the oral care compositions disclosed herein. The methods typically comprise applying any of the compositions as described herein to the teeth, e.g., by brushing, rubbing, swishing when the oral care composition is in the form of a dentifrice, or otherwise administering the oral care compositions to the oral cavity of a subject. The oral care compositions can be administered regularly, such as, for example, one or more times per day.

### EXAMPLES

### Example 1

Two non-limiting, example oral care compositions (Example Compositions A and B) were prepared in accordance with aspects of the invention. The formulation of Example Compositions A and B is shown in Table 1. Both Example Compositions A and B were in the form of a dentifrice.

TABLE 1

| | Ex. A | Ex. B |
|---|---|---|
| US INCI Name | (wt. %) | (wt. %) |
| Propylene Glycol | 62 | 61 |
| Calcium Pyrophosphate | 15 | 15 |
| Poly(ethylene glycol)-block- | 7.5 | 7.5 |
| poly(propylene glycol)-block- | | |
| poly(ethylene glycol) | | |
| Hydrogen Peroxide | 2 | 2 |
| Ammonium | 1.2 | |
| Acryloyldimethyltaurate/VP | | |
| Copolymer | | |
| Sodium | | 2 |
| Polyacryloyldimethyltaurate | | |
| Silicon Dioxide | 3 | 3 |
| Cool Flavor | 2.3 | 2.3 |
| Tetrasodium Pyrophosphate | 1.5 | 1.5 |
| Sodium Lauryl Sulfate | 2 | 2 |
| Sodium Monofluorophosphate | 0.8 | 0.8 |
| COP Sodium Saccharin | 0.7 | 0.7 |
| Sucralose | 0.05 | 0.05 |
| Butylated Hydroxoyl Toluene | 0.03 | 0.03 |
| Sodium Acid Pyrophosphate | 0.5 | 0.5 |

### Example 2

An example oral care composition (Example Composition C) was prepared in accordance with aspects of the invention. Example Composition C was in the form of a dentifrice and had the formulation shown in Table 2.

TABLE 2

| | Ex. C |
|---|---|
| US INCI Name | (wt. %) |
| Propylene Glycol | 59.71 |
| Calcium Pyrophosphate | 15 |
| Pluracare L1220 | 7.5 |
| Hydrogen Peroxide solution (50 wt. %) | 4 |
| Polyacrylate Crosspolymer-6 | 3 |
| Silicon Dioxide | 3 |
| Cool Flavor | 2.25 |
| Tetrasodium Pyrophosphate | 1.5 |
| Sodium Lauryl Sulfate | 2 |
| Sodium Monofluorophosphate | 0.76 |
| COP Sodium Saccharin | 0.7 |
| Sucralose | 0.05 |
| Butylated Hydroxoyl Toluene | 0.03 |
| Sodium Acid Pyrophosphate | 0.5 |

### Example 3

Three non-limiting, example oral care compositions (Example Compositions D and E) were prepared in accordance with aspects of the invention. A comparative composition (Comparative Composition 1) was prepared according to a similar procedure as Example Compositions D and E. Example Compositions D and E and Comparative Composition 1 were in the form of a dentifrice and had the formulations shown in Table 3.

TABLE 3

| | Ex. D | Ex. E | Comp. 1 |
|---|---|---|---|
| US INCI Name | (wt. %) | (wt. %) | (wt. %) |
| Propylene Glycol | 61.11 | 59.81 | 52.81 |
| Calcium Pyrophosphate | 15 | 15 | 15 |
| Pluracare L1220 | 7.5 | 7.5 | 7.5 |
| Hydrogen Peroxide | 2 | 2 | 2 |
| Polyvinylpyrrolidone | | 2.5 | |
| (lightly crosslinked*) | | | |
| Ammonium | 1.2 | | |
| Acryloyldimethyltaurate/VP | | | |
| Copolymer | | | |
| Polyvinylpyrrolidone | | | 9.5 |
| (crosslinked) | | | |
| Silicon Dioxide | 3.4 | 3.4 | 3.4 |
| Cool Flavor | 2.25 | 2.25 | 2.25 |
| Tetrasodium Pyrophosphate | 1.5 | 1.5 | 1.5 |
| Sodium Lauryl Sulfate | 2 | 2 | 2 |
| Sodium Monofluorophosphate | 0.76 | 0.76 | 0.76 |
| COP Sodium Saccharin | 0.7 | 0.7 | 0.7 |
| Sucralose | 0.05 | 0.05 | 0.05 |
| Butylated Hydroxoyl Toluene | 0.03 | 0.03 | 0.03 |
| Sodium Acid Pyrophosphate | 0.5 | 0.5 | 0.5 |

*The lightly crosslinked polyvinylpyrrolidone of Ex. E is less cross linked than the Polyvinylpyrrolidone of Comp. 1

### Example 4

Example Compositions D and E and Comparative Composition 1 were evaluated to assess certain properties of such compositions. Specifically, Example Compositions D and E and Comparative Composition 1 were evaluated under a standard Enamel Polishing Potential (EPP) test to assess the shine benefit of such dentifrices. The EPP values for Example Compositions D and E and Comparative Composition 1, which correspond to the compositions ability to shine a tooth, are provided in Table 4.

TABLE 4

| | Ex. D | Ex. E | Ex. F |
|---|---|---|---|
| EPP Value | 40.11 ± 3.44 | 40.21 ± 3.50 | 32.31 ± 2.49 |

As shown in Table 4, Example Compositions D and E have significantly higher EPP values than Comparative Composition 1. A higher the EPP value corresponds to a higher level of shininess/glossiness of the enamel of the tooth after brushing. Without being limited to any specific theory, it is believe that the high gelling efficiency of Example Compositions D and E enable the use of a reduced amount of polymer, which yields less coated calcium pyrophosphate particles that are more available to enamel surface.

1. An oral care composition comprising:
from about 10 to about 90 wt. % of a polyol;
a whitening agent comprising a peroxide;
from about 0.1 to about 20 wt. % of a thickening agent; and
from about 1 to about 35 wt. % of an abrasive system comprising an insoluble phosphate salt,
wherein the oral care composition contains less than about 5 wt. % of water, and all weight percentages are based on the total weight of the oral care composition.

2. The oral care composition according to claim 1, wherein the oral care composition is substantially free of or free of water.

3. The oral care composition according to claim 1, wherein the oral care composition has a total amount of all polymers of about 7 wt. % or less.

4. The oral care composition according to claim 3, wherein the total amount of polymers in the oral care composition is about 5 wt. % or less.

5. The oral care composition according to claim 1, wherein the polyol is present in an amount from about 50 to about 90 wt. %.

6. The oral care composition according to claim 1, wherein the polyol comprises a C₂-C₃₂ polyol.

7. The oral care composition according to claim 1, wherein the polyol is selected from ethylene glycol, propylene glycol, butylene glycol, hexylene glycol, glycerin, diglycerin, diethylene glycol, dipropylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol, and a combination of two or more thereof.

8. The oral care composition according to claim 1, wherein the peroxide is selected from hydrogen peroxide, lithium peroxide, potassium peroxide, sodium peroxide, magnesium peroxide, calcium peroxide, barium peroxide, alkyl hydrogen peroxides, dialkyl peroxides, alkyl peroxy acids, peroxy esters, diacyl peroxides, benzoyl peroxide, and monoperoxyphthalate, and a combination of two or more thereof.

9. (canceled)

10. The oral care composition according to claim 1, wherein the peroxide is selected from PVP complexed with hydrogen peroxide, silica complexed with hydrogen peroxide, tetrasodium pyrophosphate complexed with hydrogen peroxide, and a combination thereof.

11. (canceled)

12. The oral care composition according to claim 1, wherein the thickening agent comprises an ammonium acryloyldimethyltaurate/VP copolymer, a sodium polyacryloyldimethyltaurate, polyvinylpyrrolidone, cross-polymer 6, a silica thickener, or a combination of two or more thereof.

13-14. (canceled)

15. The oral care composition according to claim 1, further comprising an anti-calculus agent, optionally, in an amount from about 0.5 to about 10 wt. %.

16. The oral care composition according to claim 15, wherein the anti-calculus agent is selected from tetrasodium pyrophosphate, ammonium tripolyphosphate, hexametaphosphate salt, polycarboxylate polymers, polyvinyl methyl ether/maleic anhydride (PVME/MA) copolymers, and a combination of two or more thereof.

17-18. (canceled)

19. The oral care composition according to claim 1, further comprising a fluoride source, optionally, in an amount from about 0.1 to about 2 wt. %.

20. The oral care composition according to claim 19, wherein the fluoride source is selected from stannous fluoride, sodium fluoride, potassium fluoride, sodium monofluorophosphate, sodium fluorosilicate, ammonium fluorosilicate, amine fluoride, ammonium fluoride, and a combination of two or more thereof.

21. The oral care composition according to claim 1, further comprising an anionic surfactant, optionally, in an amount from about 0.5 to about 10 wt. %.

22. The oral care composition according to claim 21, wherein the anionic surfactant is selected from ammonium lauryl sulfate, ammonium lauryl ether sulfate, sodium dodecyl sulfate, sodium coco-sulfate, ammonium coco-sulfate, sodium lauryl sulfate, sodium lauryl ether sulfate, and a combination of two or more thereof.

23. The oral care composition according to claim 1, further comprising a non-ionic surfactant, optionally, in an amount from about 0.5 to about 10 wt. %.

24. The oral care composition according to claim 1, further comprising potassium nitrate.

25. (canceled)

26. The oral care composition according to claim 1, wherein the peroxide is a non-complexed peroxide present in the amount of from about 0.01 to about 10 wt. %.

27. An anhydrous oral care composition comprising:
a polyol comprising propylene glycol;
a whitening agent comprising hydrogen peroxide;
a thickening system comprising an ammonium acryloyldimethyltaurate/VP copolymer, a sodium acryloyldimethyltaurate, or a combination of two or more thereof; and
an abrasive system comprising an insoluble phosphate salt,
wherein the oral care composition has about 7 wt. % or less of a total amount of polymers, based on the total weight of the oral care composition.
